(19) United States
(12) Patent Application Publication
(10) Pub. No.: US 2009/0258879 A1
(43) Pub. Date: Oct. 15, 2009

(54) METHOD FOR TREATING CANCER, RHEUMATOID ARTHRITIS AND OTHER MEDICAL DISEASES BY UTILIZING MODIFIED VIRUS VIRIONS TO INSERT MEDICATIONS INTO TARGETED CELLS

(76) Inventors: Lane Bernard Scheiber, Annandale, VA (US); Lane Bernard Scheiber, II, Grosse Ile, MI (US)

Correspondence Address: LANE B. SCHEIBER, 7864 OAK RIVER DRIVE, GROSS ILE, MI, 48138, US

(21) Appl. No.: 12/101,966

(22) Filed: Apr. 12, 2008

(51) Int. Cl. A61K 31/517; A61P 19/02

(52) U.S. Cl. 514/249

(57) ABSTRACT

A safer, more effective treatment of many medical diseases may be approached by a method utilizing modified viruses as vehicles to transport medically therapeutic drug molecules to specific cells in the body with the intent to have the drug exert an effect only on those cells to which the modified virus delivers the drug. The modified virus or virus-like structures make contact with specific target cells by means of the modified virus's exterior probes or virus-like structures' exterior probes. Once the exterior probes engage a target cell's receptors, the modified virus or virus-like structure inserts into the target cell the quantity of medically therapeutic drug molecules it is carrying. By delivering the medically therapeutic drug only to specific cells in the body it is assured the drug reaches the site in the body it will be most beneficial and the occurrence of unwanted side effects due the drug are significantly minimized.

### CROSS REFERENCE TO RELATED APPLICATIONS

None.

### STATEMENT REGARDING SPONSORED RESEARCH OR DEVELOPMENT

None.

### REFERENCE TO SEQUENCE LISTING, A TABLE, OR COMPUTER LISTING COMPACT DISC APPENDIX

Not applicable.

©2008 Lane B. Scheiber and Lane B. Scheiber II. A portion of the disclosure of this patent document contains material which is subject to copyright protection. The copyright owner has no objection to the facsimile reproduction by anyone of the patent document or the patent disclosure, as it appears in the Patent and Trademark Office patent file or records, but otherwise reserves all copyright rights whatsoever.

### BACKGROUND OF THE INVENTION

1. Field of the Invention

This invention relates to any medical treatment method intended to treat a medical condition in the body by utilizing a modified virus to insert a drug into specific cells of the body.

2. Description of Background Art

Treatment of cancer, rheumatoid arthritis and other medical conditions may be approached by a method utilizing modified virus virions as vehicles to transport medically therapeutic drugs to cells to increase the potency of the drug and significantly reduce the deleterious side effects of the drug. An intact, individual form of a virus, as it exists outside the boundaries of a host cell, is generally referred to as a ‘virion’.

The current approach medical drug treatment to manage cancer generally involves administering chemotherapy to an individual afflicted with cancer. The intention of administering the chemotherapy is in theory to poison and kill the cancer cells before causing significant side effects to the individual receiving the chemotherapy. Cancer cells generally grow and multiply at a rate that is faster than normal cells. The side effects patients experience from chemotherapy is often related to the harmful effects of the chemotherapy poisoning the healthy cells of the body as well as the cancer cells.

One of the most successful approaches to the treatment of rheumatoid arthritis in the last two decades has been the use of a chemotherapy known as methotrexate. Methotrexate is administered to patients either orally or by injection. Methotrexate interferes with the folate metabolism in cells, which results in decreased cellular metabolism and inhibits cellular replication. Methotrexate tends to exert its effect on cells that are growing and multiplying faster than normal. Cancer cells and in rheumatoid arthritis, synovial cells, grow and multiply at a rate that is faster than normal cellular metabolism. In rheumatoid arthritis, low dose methotrexate tends to inhibit the growth of the synovial cells that surround the joints and tendons in the body. The inhibition of synovial cell proliferation results in the disease being put into a state of remission and the crippling effects of the disease being averted.

Many drugs used to treat medical diseases have limited success and incomplete compliance by patients due to the fact that drugs often cause unwanted side effects when healthy cells suffer delirious effects of the drug. A drug introduced into the body by means of an oral route, inhaled route, rectal suppository or an injectable manner may affect every cell it comes in contact with rather than limiting its effects on the specific tissues or specific cells that the drug is intended to exert an effect on to generate a medically therapeutic outcome. Adverse side effects generated by systemic effects of drugs may be minimized by limiting the delivery of a drug to specific target cells or specific tissues in the body.

A eukaryote refers to a nucleated cell. Eukaryotes comprise nearly all animal and plant cells. A human eukaryote or nucleated cell is comprised of an exterior lipid bilayer plasma membrane, cytoplasm, a nucleus, and organelles. The exterior plasma membrane defines the perimeter of the cell, regulates the flow of nutrients, water and regulating molecules in and out of the cell, and has embedded into its structure cell-surface receptors that the cell uses to detect properties of the environment surrounding the cell membrane. Cytoplasm refers to the entire contents inside the cell except for the nucleus and acts as a filling medium inside the boundaries of the plasma cell membrane. Cytosol refers to the semifluid portion of the cytoplasm minus the mitochondria and the endoplasmic reticulum. The nucleus, organelles, and ribosomes are suspended in the cytosol. Nutrients such as amino acids, oxygen and glucose are present in the cytosol. The nucleus contains the majority of the cell's genetic information in the form of double stranded deoxyribonucleic acid (DNA). Organelles generally carry out specialized functions for the cell and include such structures as the mitochondria, the endoplasmic reticulum, storage vacuoles, lysosomes and Golgi complex. Floating in the cytoplasm, but also located in the endoplasmic reticulum and mitochondria are ribosomes. Ribosomes are protein structures comprised of several strands of proteins that combine and couple to a messenger ribonucleic acid (mRNA) molecule. More than one ribosome may be attached to a single mRNA at a time. Ribosomes decode genetic information coded in a mRNA molecule and manufacture proteins to the specifications of the instruction code physically present in the mRNA molecule.

The majority of the deoxyribonucleic acid (DNA) in a cell is present in the form of chromosomes, the double stranded helical structures located in the nucleus of the cell. DNA in a circular form, can also be found in the mitochondria, the powerhouse of the cell, an organelle that assists in converting glucose into usable energy molecules. DNA represents the genetic information a cell needs to manufacture the materials it requires to develop to its mature form, sustain life and to replicate. Genetic information is stored in the DNA by arrangements of four nucleotides referred to as: adenine, thymine, guanine and cytosine. DNA represents instruction coding, that in the process known as transcription, the DNA's genetic information is decoded by transcription protein complexes referred to as polymerases, to produce ribonucleic acid (RNA). RNA is a single strand of genetic information comprised of coded arrangements of four nucleotides: adenine, uracil, guanine and cytosine. The physical difference in the construction of a DNA molecule versus a RNA molecule is that DNA utilizes the nucleotide ‘thymine’, while RNA molecules utilize the nucleotide ‘uracil’. RNAs are generally classified as messenger RNAs (mRNA), transport RNAs (tRNA) and ribosomal RNAs (rRNA).

Proteins are comprised of a series of amino acids bonded together in a linear strand, sometimes referred to as a chain; a protein may be further modified to be a structure comprised of one or more similar or differing strands of amino acids bonded together. A protein comprised of one or more strands of amino acids (referred to as subunits) may be referred to as a protein complex. Insulin is a protein structure comprised of two strands of amino acids, one strand comprised of 21 amino acids long and the second strand comprised of 30 amino acids; the two strands attached by two disulfide bridges. There are an estimated 30,000 different proteins the cells of the human body may manufacture. The human body is comprised of a wide variety of cells, many with specialized functions requiring unique combinations of proteins and protein structures such as glycoproteins (a protein combined with a carbohydrate) to accomplish the required task or tasks a specialized cell is designed to perform. Forms of glycoproteins are known to be utilized as cell-surface receptors.

Viruses are obligate parasites. Viruses simply represent a carrier of genetic material and by themselves viruses are unable to replicate or carry on any form of biologic function outside their host cell. Viruses are generally comprised of one or more shells constructed of one or more layers of protein or lipid material, and inside the outer shell or shells, a virus carries a genetic payload that represents the instruction code necessary to replicate the virus, and protein enzymes to help facilitate the genetic payload in the function of replicating copies of the virus once the genetic payload has been delivered to a host cell. Located on the outer shell or envelope of a virus are probes. The function of a virus's probes is to locate and engage a host cell's receptors. The virus's surface probes are designed to detect, make contact with and functionally engage one or more receptors located on the exterior of a cell type that will offer the virus the proper environment in which to construct copies of itself. A host cell is a cell that provides the virus the proper biochemical machinery for the virus to successfully replicate itself.

Protected by the outer coat generally comprised of an envelope or capsid or envelope and capsid, viruses carry a genetic payload in the form of deoxyribonucleic acid (DNA) or ribonucleic acid (RNA). Once a virus's exterior probes locate and functionally engage the surface receptor or receptors on a host cell, the virus inserts its genetic payload into the interior of the host cell. In the event a virus is carrying a DNA payload, the virus's DNA travels to the host cell's nucleus and is known to become inserted into the host cell's own native DNA. In the case where a virus is carrying its genetic payload as RNA, the virus inserts the RNA payload into the host cell and may also insert one or more enzymes to facilitate the RNA being utilized properly to replicate copies of the virus. Once inside the host cell, some species of virus facilitate their RNA being converted to DNA. Once the viral RNA has been converted to DNA, the virus's DNA travels to the host cell's nucleus and is known to become inserted into the host cell's native DNA. Once a virus's genetic material has been inserted into the host cell's native DNA, the virus's genetic material takes command of certain cell functions and redirects the resources of the host cell to generate copies of the virus. Other forms of RNA viruses bypass the need to use the host cell's nuclear DNA and simply utilize portions of its innate viral genome to act as messenger RNA (mRNA). RNA viruses that bypass the host cell's DNA, cause the cell, in general, to generate copies of the necessary parts of the virus directly from the virus's RNA genome. When a virus's genome directly acts as a template, then similar to the cell's messenger RNA, the virus's RNA is read by the cell's ribosomes and proteins necessary to complete the virus's replication process are generated.

The Hepatitis C virus (HCV) is a positive sense RNA virus, meaning a type of RNA that is capable of bypassing the need for involving the host cell's nucleus by having its RNA genome function as messenger RNA. Hepatitis C virus infects liver cells. The Hepatitis C viral genome becomes divided once it gains access to the interior of a liver host cell. Portions of the subdivisions of the Hepatitis C viral genome directly interact with host liver cell's ribosomes to produce proteins necessary to construct copies of the virus.

HCV belongs to the Flaviviridae family and is the only member of the Hepacivirus genus. There are considered to be at least 100 different strains of Hepatitis C virus based on genome sequencing variability.

HCV is comprised of an outer lipoprotein envelope and an internal nucleocapsid. The genetic payload is carried within the nucleocapsid. In its natural state, present on the surface of the outer envelope of the Hepatitis C virus are probes that detect receptors present on the surface of liver cells. The glycoprotein E1 probe and the glycoprotein E2 probe have been identified to be affixed to the surface of HCV. The E2 probe binds with high affinity to the large external loop of a CD81 cell-surface receptor. CD81 is found on the surface of many cell types including liver cells. Once the E2 probe has engaged the CD81 cell-surface receptor, cofactors on the surface of HCV's exterior envelope engage either or both the low density lipoprotein receptor (LDLR) or the scavenger receptor class B type I (SR-BI) present on the liver cell in order to activate the mechanism to facilitate HCV breaching the cell membrane and inserting its RNA genome payload through the plasma cell membrane of the liver cell into the liver cell. Upon successful engagement of the HCV surface probes with a liver cell's cell-surface receptors, HCV inserts the single strand of RNA and other payload elements it carries into the liver cell targeted to be a host cell. The HCV RNA genome then interacts with enzymes and ribosomes inside the liver cell in a translational process to produce the proteins required to construct copies of the protein components of HCV. The HCV genome undergoes a method of transcription to replicate copies of the virus's RNA genome. Inside the host, pieces of the HCV virus are assembled together and ultimately loaded with a copy of the HCV genome. Replicas of the original HCV then escape the host cell and migrate the environment in search of additional host liver cells to infect and continue the replication process.

The HCV's naturally occurring genetic payload consists of a single molecule of linear positive sense, single stranded RNA approximately 9600 nucleotides in length. By means of a translational process a polyprotein of approximately 3000 amino acids is generated. This polyprotein is cleaved post translation by host and viral proteases into individual viral proteins which include: the structural proteins of C, E1, E2, the nonstructural proteins NS1, NS2, NS3, NS4A, NS4B, NS5A, NS5B, p7 and ARFP/F protein. Hepatitis C virus's proteins direct the host liver cell to construct copies of the Hepatitis C virus. A membrane associated replicase complex consisting of the virus's nonstructural proteins NS3 and NS5B facilitate the replication of the viral genome. The membrane of the endoplasmic reticulum appears to be the site of protein maturation and viral assembly. Once copies of the Hepatitis C Virus are generated, they exit the host cell and each copy of HCV migrates in search of another appropriate liver cell that will act as a host to continue the replication process.

Hepatitis C virus offers a naturally occurring vehicle mechanism to transport and insert medically therapeutic drug molecules into liver cells and other specifically targeted cells of the human body. The naturally occurring Hepatitis C virus already is equipped with the means of seeking out liver cells and delivering to liver cells its genetic payload. Further, the surface probes present on the Hepatitis C virus's outer protein coat can be modified to seek out specific receptors on specific target cells. HCV's innate genetic payload could be replaced by a payload consisting of medically therapeutic drug molecules. Once the modified Hepatitis C virus's probes properly engage the cell-surface receptors on a target cell, the modified Hepatitis C virus would insert into the target cell a medically therapeutic drug for the purpose of achieving a medical therapeutic response.

The Hepatitis C virus is one of several viruses that have been identified that possess the natural capacity to locate and infect liver cells with the genome the virus carries, thus including a liver cell as part of its reproductive cycle. Hepatitis A virus (HAV), Hepatitis C virus (HCV), Hepatitis D virus (HDV), Hepatitis E virus (HEV), and Hepatitis G virus (HGV) have been identified to carry their genome as RNA. The Hepatitis G virus is considered to be very similar to the Hepatitis C virus. The Hepatitis F virus and Hepatitis H viruses at this point are not considered to exist, though this is controversial. The Hepatitis B virus (HBV) is believed to carry its genome as DNA. These alternative hepatitis viruses may also be utilized to act as alternative vehicles to deliver medically therapeutic drug molecules to liver cells or specific target cells.

Current state of gene therapy generally refers to efforts directed toward inserting an exogenous subunit of DNA into a vehicle such as a naturally occurring virus. The vehicle is intended to insert the exogenous subunit of DNA into a target cell. The exogenous DNA subunit then migrates to the target cell's nucleus. The exogenous DNA subunit then inserts into the native DNA of the cell. This represents a permanent alteration of the cell's nuclear DNA. At some point, the nuclear transcription proteins read the exogenous DNA subunit's nucleotide coding to produce the intended cellular response. The approach described within the scope of this text involves a medically therapeutic drug as a payload versus DNA or RNA as a payload. DNA is comprised of the nucleotides adenine, thymine, guanine and cytosine. RNA is composed of the nucleotides adenine, uracil, guanine and cytosine. DNA codes acts as a template to code for the manufacture of RNA molecules. RNA acts as a template coding for the manufacture of proteins, which are composed of amino acids. A drug acts to function as a participant in a chemical reaction, as either a catalyst of the reaction or with another substance to produce one or more additional substances, these additional substances often having different properties. The virus chosen as the example of a transport vehicle, Hepatitis C virus, could be outfitted to carry a medically therapeutic drug molecules rather than RNA or DNA molecules.

Treatment of cancer may be approached by a method utilizing modified viruses as vehicles to transport medically therapeutic drugs directly to cancer cells with the intent to directly poison cancer cells by having the drug the modified virus virions carry interfere with the metabolism of the cancer cells. Forms of liver cancer, including primary cancers of the liver or secondary cancers due to metastasis, can be treated utilizing modified hepatitis viruses to carry chemotherapy directly to liver cells to terminate cancer cells residing in the liver.

Treatment of rheumatoid arthritis may be approached by a method utilizing modified virus virions as vehicles to transport a medically therapeutic drug such as methotrexate directly to the synovial cells associated with joints and tendons with the intent to have the drug interfere with the metabolism of the synovial cells in order to place the disease in remission. Liver and bone marrow toxicity is often an unwanted side effect of methotrexate therapy. To be able to deliver the methotrexate directly to the synovial cells without exposing the cells of the body as a whole to the effects of methotrexate, would significantly decrease the side effects of the drug and provide an effective therapy to many more patients than are currently able to tolerate the drug. In addition delivering glucosamine molecules and chondroitin molecules to chondrocytes, the cells responsible for producing cartilage, will increase cartilage production on the surface of bones. Delivering nonsteroidal anti-inflammatory drug molecules directly to synovial cells and muscle cells will reduce the inflammation associated with arthritis.

A safer, more effective treatment of many disease may be approached by a method utilizing modified virus virions as vehicles to transport medically therapeutic drug molecules to specific cells in the body with the intent to have the drug exert an effect only on those cells to which the modified virus virions deliver the drug.

### BRIEF SUMMARY OF THE INVENTION

The method by which a quantity of modified virus virions or virus-like structures are used as a transport medium to carry a payload consisting of a quantity of medically therapeutic drug molecules to specific cells in the body. The modified virus virions or virus-like structures make contact with specific target cells by means of the modified virus virions' exterior probes or virus-like structures' exterior probes. Once the exterior probes engage the target cells' receptors, the modified virus virions or virus-like structures insert into the target cells the quantity of medically therapeutic drug molecules they are carrying.

### DETAILED DESCRIPTION

Viruses or virus-like structures can be fashioned to act as transport vehicles to carry and deliver medically therapeutic drug molecules directly to specific cells. The medically therapeutic drug carried by therapeutic modified viruses or virus-like structures would supply the cells of the body with the drug without interfering or harming other cells in the body.

Naturally occurring viruses can be altered by replacing the genetic material the virus would carry, with medically therapeutic drug molecules that would have a beneficial medically therapeutic effect on cells. The naturally occurring virus would then carry and deliver to its natural target cell the payload of medically therapeutic drug molecules. As an example, hepatitis viruses could be altered to carry medically therapeutic drug molecules to liver cells. The naturally occurring virus then, instead of causing disease associated with delivering its own genome to conduct its replication process, would instead act as a method to deliver a quantity of medically therapeutic drug molecules, which would provide the target cell with a medically therapeutic outcome.

Naturally occurring viruses can be further modified to have their naturally occurring glycoprotein surface probes replaced by glycoprotein surface probes that target specific cells in the body. Viruses modified to carry and deliver medically therapeutic drug molecules as the payload, further modified to have their glycoprotein surface probes, that cause the modified virus to engage specific cells in the body, provides a method whereby specific cells in the body can be targeted and this method embodies a means of providing to a specific type of cell in the body a drug to participate in chemical reactions with the intent to accomplish a medically therapeutic outcome.

Virus-like structures can be constructed with similar physical characteristics to naturally occurring viruses and be fashioned to carry medically therapeutic drug molecules as the payload and have located on the surface glycoprotein probes that engage specific cells in the body. Viruses-like structures carrying medically therapeutic drug molecules as the payload, constructed to have their glycoprotein surface probes engage specific cells in the body, and deliver to those specific cells the drug the virus-like structures carry provides a method whereby specific cells in the body can be targeted and this method embodies a means of providing to a specific type of cell in the body a drug to participate in chemical reactions with the intent to accomplish a medically therapeutic outcome. The advantage of a virus-like structure is that the physical dimensions of the virus-like structure can be adjusted to accommodate variations in the physical size of the payload of medically therapeutic drug molecules, yet maintain a means of engaging targeted cells in the body and delivering to those targeted cells the drug molecules required to accomplish the desired medical therapeutic outcome. A second advantage of utilizing virus-like structures is to be able to change the surface characteristics of the transport vehicle to prevent the body's immune system from reacting to the presence of the therapeutic modified virus and destroying the modified virus before it is able to deliver the payload it carries to the cells it has been designed to target. HIV utilizes an exterior envelope comprised of the surface membrane of its host, the T-Helper cell, which acts as a disguise to fool the body's immune system detection resources. Virus-like structures could be fashioned, similar to HIV, to have as an exterior envelope a surface that resembles a cell's outer membrane. Constructing virus-like structures with an exterior envelope that resembles a cell's outer membrane would assist in the virus-like structure being able to avoid detection by the body's immune system to improve survivability of the virus-like structure thus improving the virus-like structures' chances of reaching the cells it is targeted for and delivering to those cells the drug that it carries as a payload.

The Hepatitis C virus virion provides a naturally occurring specimen to illustrate the feasibility of the method described in this text. The Hepatitis C virus (HCV) virion is comprised of an outer lipoprotein envelope and an internal nucleocapsid. The virus's genetic payload is carried within its core, the nucleocapsid. The HCV's naturally occurring genetic payload consists of a single molecule of linear positive sense, single stranded RNA approximately 9600 nucleotides in length, which includes: the structural proteins of C, E1, E2, the nonstructural proteins NS1, NS2, NS3, NS4A, NS4B, NS5A, NS5B, p7 and ARFP/F protein. Present on the surface of the outer envelope of the Hepatitis C virus virion are probes that detect receptors present on the surface of liver cells. The glycoproteins E1 and E2 have been identified to be affixed to the surface of HCV virion. Portions of the Hepatitis C virus genome, when separated into individual pieces, behave like messenger RNA. The naturally occurring HCV virion is constructed with surface probes fashioned to recognize receptors on the surface of a liver cell. Once the naturally occurring HCV's surface probe E2 engages a liver cell's CD81 receptor, and cofactors on the surface of HCV's exterior envelope engage the low density lipoprotein receptor (LDLR) or the scavenger receptor class B type I (SR-BI) on the liver cell, the HCV virion then has the opportunity to insert its RNA genetic payload into the engaged target liver cell.

The Hepatitis C virus virion carrying an mRNA payload, infects liver cells with its payload for the purpose of causing the now infected cell to generate a variety of proteins that will be assembled into copies resembling the original HCV virion. The copies of the HCV virion are then released from the infected cell to migrate in search of other host cells. Variations in the Hepatitis C virus are based on variations that occur in the strand of mRNA molecule the HCV virion carries as it genome. HCV virions may therefore carry differing mRNA molecules as its genetic payload and deliver these mRNA molecules specifically to liver cells in the body to cause these cells to produce proteins to accomplish the task of replication of similar HCV virions.

Replicating viruses and constructing viruses to carry DNA payloads is a form of manufacturing technology that has already been well established and is in use facilitating the concept of gene therapy. Replicating viruses and designing these viruses to carry drug as the genetic payload would incorporate similar techniques as already proven useful in current DNA gene therapy technologies.

To carry out the process to manufacture a modified medically therapeutic Hepatitis C virus, messenger RNA that would code for the general physical outer structures of the Hepatitis C virus would be inserted into a host. The host may include devices such as a host cell or a hybrid host cell. The host may utilize DNA or RNA or a combination of genetic instructions in order to accomplish the construction of medically therapeutic modified virus virions. The DNA or messenger RNA molecules to create the medically therapeutic hepatitis virus would direct the cells to generate copies of the medically therapeutic virus carrying a medically therapeutic drug payload. In some cases DNA or messenger RNA would be inserted into the host that would be coded to cause the production of surface probes that would be affixed to the surface of the virus virion that would target the surface receptors on specific cells in the body other than the liver cells the Hepatitis C virus naturally targets. DNA or messenger RNA would direct the host to generate copies of the medically therapeutic drug molecules that would provide a therapeutic action, or alternatively the medically therapeutic drug molecules would be artificially introduced into the host; these medically therapeutic drug molecules would take the place of the Hepatitis C virus's innate genome as its payload. The medical treatment form of the Hepatitis C virus carrying the medically therapeutic drug molecules would be produced, assembled and released from a host. Virus-like structures would be generated in similar fashion using a host such as host-cells or hybrid host cells. The copies of the medically therapeutic hepatitis virus or virus-like structures, upon exiting the host, would be collected, stored and utilized as a medical treatment as necessary.

The modified Hepatitis C virus and virus-like structures would be incapable of replication on its own due to the fact that the messenger RNA that would code for the replication process to produce copies of the virus or virus-like structure would not be present in the modified form of the Hepatitis C virus or virus-like structure.

In review, the method described in this text includes taking a naturally occurring virus and altering its payload so that it transports medically therapeutic drug molecules to cells it was naturally designed to infect, but instead of delivering its own genetic payload, it delivers the medically therapeutic drug molecules it is carrying, and the method described in this text includes taking a naturally occurring virus and altering its payload so that it carries medically therapeutic drug molecules to cells and alter the virus's glycoprotein probes so that it is capable of infecting specifically targeted cells, but instead of delivering its own genetic payload, it delivers the medically therapeutic drug molecules it is carrying to specific target cells, and the method described in this text includes taking a virus-like structure, which carries medically therapeutic drug molecules to cells, affixed to the surface glycoprotein probes so that it is capable of delivering medically therapeutic drug molecules it is carrying to specific target cells.

As mentioned above, a quantity of modified virus virions, such as Hepatitis C virus virions would be introduced into a patient's blood stream or tissues so that the modified virus could deliver the medially therapeutic drug payload that it carries to targeted cells in the body, such as liver cells.

The medical treatment method will treat medical diseases associated with a variety of cell types including cells comprising a cancer, cells comprising a malignancy, cells comprising a tumor, cells comprising synovial tissues surrounding a joint, cells comprising synovial tissues surrounding a tendon, cells comprising synovial tissues surrounding a rheumatoid nodule, cells comprising the muscles, cells comprising the brain, cells comprising the heart, cells comprising the pancreas, cells comprising the endocrine glands, cells comprising the dermis, cells comprising the mucosa, cells comprising the gastroenteric tract, cells comprising the renal system, cells comprising the skeletal structure, cells comprising the pulmonary system, cells comprising the nervous system, cells comprising the immune system, cells comprising the sex organs, cells comprising the connective tissues, cells comprising the spleen, cells comprising the eyes, cells comprising the reticuloendothelial system, and cells comprising the liver.

By utilizing the described method to provide the cells of the body with the above-mentioned medically therapeutic drug molecules and enhancing the capacity of cells to treat a variety of medical conditions from cancer to rheumatoid arthritis, which will result the betterment of medical management for patients.

### DRAWING

None.

What is claimed:

1. A medical treatment method for inserting a quantity of medically therapeutic drug molecules into cells of the body comprising:
(a) a quantity of modified virus virions generated for the purpose of transporting a quantity of said medically therapeutic drug molecules,
(b) the quantity of said modified virus virions having a quantity of glycoprotein probes affixed to their surface, said glycoprotein probes constructed in a manner to target specific cells in said body,
(c) said glycoprotein probes capable of engaging specific cell-surface receptors on said cells,
(d) once said glycoprotein probes have successfully engaged cell-surface receptors on said cells, the quantity of said modified virus virions deliver into said cells a quantity of said medically therapeutic drug molecules said modified virus virions are carrying,
whereby the chemical action of said medically therapeutic drug molecules will be available in said cells for the purpose of treating a medical disease,
whereby the adverse side effects of said medically therapeutic drug molecules will be minimized by the action of said drug being delivered specifically to said cells that would benefit from the presence of said drug rather than exposing the cells of the body as a whole to the effects of said medically therapeutic drug molecules,
whereby to achieve a therapeutic effect of said drug a much lower dose of said drug in comparison to conventional dosing will be required due to said drug molecules being delivered directly to said cells that would benefit from the chemical reaction said drug would participate in while present in said cells resulting in a lower frequency of drug toxicity experienced by said body.

2. The medical treatment method in claim 1 wherein said modified virus virions selected from the group consisting of naturally occurring virus virions whose payload has been altered to carry a quantity of medically therapeutic drug molecules, naturally occurring virus virions whose payload has been altered to carry a quantity of medically therapeutic drug molecules said virus virions capable of delivering said quantity of medically therapeutic drug molecules which the surface glycoprotein probes have been altered in a manner the glycoprotein probes are fashioned to engage specific cells in said body, and virus-like structures constructed to resemble naturally occurring virus virions said virus-like structures capable of carrying a quantity of medically therapeutic drug molecules said virus-like structures constructed with glycoprotein probes fashioned to engage specific cells in said body said virus-like structures capable of delivering said quantity of medically therapeutic drug molecules to said specific cells in said body.

3. The medical treatment method in claim 1 wherein said specific cells selected from the group consisting of cells comprising a cancer, cells comprising a malignancy, cells comprising a tumor, cells comprising synovial tissues surrounding a joint, cells comprising synovial tissues surrounding a tendon, cells comprising synovial tissues surrounding a rheumatoid nodule, cells comprising the muscles, cells comprising the brain, cells comprising the heart, cells comprising the pancreas, cells comprising the endocrine glands, cells comprising the dermis, cells comprising the mucosa, cells comprising the gastroenteric tract, cells comprising the renal system, cells comprising the skeletal structures, cells comprising the pulmonary system, cells comprising the nervous system, cells comprising the immune system, cells comprising the sex organs, cells comprising the connective tissues, cells comprising the spleen, cells comprising the eyes, cells comprising the reticuloendothelial system, and cells comprising the liver.

4. The medical treatment method in claim 1 wherein said medically therapeutic drug molecules selected from a group consisting of chemotherapy drug molecules, methotrexate drug molecules, glucosamine molecules, chondroitin molecules and nonsteroidal anti-inflammatory drug molecules.

5. The medical treatment method in claim 1 wherein said body is comprised of the physical features of the human body.

6. A medical treatment method for inserting a quantity of medically therapeutic drug molecules into liver cells of the body comprising:
(a) a quantity of modified virus virions generated for the purpose of transporting a quantity of said medically therapeutic drug molecules,
(b) the quantity of said virus virions having a quantity of glycoprotein probes affixed to their surface, said glycoprotein probes constructed in a manner to target said liver cells in said body,
(c) said glycoprotein probes capable of engaging specific cell-surface receptors on said liver cells,
(d) once said glycoprotein probes have successfully engaged cell-surface receptors on said liver cells, the quantity of said modified virus virions deliver into said liver cells a quantity of said medically therapeutic drug molecules said modified virus virions are carrying,
whereby the chemical action of said medically therapeutic drug molecules will be available in said liver cell to for the purpose of treating a medical disease,
whereby the adverse side effects of said medically therapeutic drug molecules will be minimized by the action of said drug being delivered specifically to said liver cells that would benefit from the presence of said drug rather than exposing the cells of the body as a whole to the effects of said medically therapeutic drug molecules,
whereby to achieve a therapeutic effect of said drug a much lower dose of said drug in comparison to conventional dosing will be required due to said drug molecules being delivered directly to said liver cells that would benefit from the chemical reaction said drug molecules would participate in while present in said liver cells resulting in a lower frequency of drug toxicity experienced by said body.

7. The medical treatment method in claim 6 wherein said modified virus virions selected from the group consisting of Hepatitis A virus virions, Hepatitis B virus virions, Hepatitis C virus virions, Hepatitis D virus virions, Hepatitis F virus virions, Hepatitis E virus virions, Hepatitis G virus virions, and Hepatitis H virus virions.

8. The medical treatment method in claim 6 wherein said modified virus virions selected from the group consisting of naturally occurring virus virions whose payload has been altered to carry a quantity of medically therapeutic drug molecules, naturally occurring virus virions whose payload has been altered to carry a quantity of medically therapeutic drug molecules said virus virions capable of delivering said quantity of medically therapeutic drug molecules which the surface glycoprotein probes have been altered in a manner the glycoprotein probes are fashioned to engage liver cells in said body, and virus-like structures constructed to resemble naturally occurring virus virions said virus-like structures capable of carrying a quantity of medically therapeutic drug molecules said virus-like structures constructed with glycoprotein probes fashioned to engage liver cells in said body said virus-like structures capable of delivering said quantity of medically therapeutic drug molecules to said liver cells in said body.

9. The medical treatment method in claim 6 wherein said medically therapeutic drug molecules are chemotherapy drug molecules.

10. The medical treatment method in claim 6 wherein said body is comprised of the physical features of the human body.
